(19) United States
(12) Patent Application Publication
(10) Pub. No.: US 2013/0046261 A1
(43) Pub. Date: Feb. 21, 2013

(54) Decorative Adhesive Wound Care Systems

(71) Applicant: Dion R. Reif, Petaluma, CA (US)

(72) Inventor: Dion R. Reif, Petaluma, CA (US)

(21) Appl. No.: 13/282,886

(22) Filed: Oct. 27, 2011

Related U.S. Application Data
(60) Provisional application No. 61/539,391, filed on Sep. 26, 2011.

(51) Int. Cl. A61L 15/42; A61F 13/02; A61F 13/15

(52) U.S. Cl. 604/367; 604/385.01

(57) ABSTRACT

Adhesive bandages for wound covering and care have an external surface adapted to receive ink or coloration from a drawing media, such as a pen, pencil and marker, without the coloration penetrating beyond the outer surface or to the would care absorbent and protective matter. Other aspects of the invention include a kit comprising such bandage device, along with suitable drawing media. In a method of use, the bandage devices can be marked before or after application to the wound. The outer surface of the adhesive bandage that is intended to be colored by the drawing media may be pre-printed with drawing/marking guides or outlines that are attractive to young children, such as cartoon characters, animals, superheroes and the like.

### CROSS REFERENCE TO RELATED APPLICATIONS

The present application claims the benefit of priority to the US Provisional Patent Application of the same title that was filed on Sep. 26, 2011, having application Ser. No. 61/539,391, which is incorporated herein by reference.

### BACKGROUND OF INVENTION

The present invention relates to bandages and external wound care cover devices, and in particular to such products capable of decorative enhancement without compromise to the therapeutic performance.

Prior bandages with decorative enhancements are disclosed in U.S. Pat. No. 4,094,316 issued to Nathanson issued Jun. 13, 1978 for an ADHESIVE BANDAGE WITH REUSABLE APPLIQUE; U.S. Pat. No. 6,455,752 BI issued to Vesey on Sep. 24, 2002 for a “DECORATIVE ADHESIVE BANDAGE KIT”, and US Patent Application Publication No. US 2005/0101899 A1 (Wegmann) on May 12, 2005 for a BAND AID APPLIQUE, all of which are incorporated herein by reference.

However, it is believed that such devices have various short comings, in particular with respect to being suitable for use by young children and drawing their interest and attention to distract them long enough from the injury that requires wound care.

Hence, an object of the present invention are therefore to provide new bandages and wound care systems that provide a distracting activity for an injured child, aesthetic enjoyment, as well as an incentive to cooperate in the process of therapeutic wound care.

### SUMMARY OF INVENTION

In the present invention, the first object is achieved by providing a bandage comprising: a generally thin planar and flexible absorbent layer having an upper surface and an opposing lower surface, a generally thin planar and flexible base layer having an outer surface and an opposing inner surface, wherein said base layer laterally expands beyond at least two edges of the absorbent layer, and further wherein the upper surface of the said absorbent layer is attached the inner surface of the base layer, a generally thin planar and flexible adhesive layer attached to and covering the inner portions of the base layer that is not attached to the absorbent layer, wherein the outer surface of said base layer opposite is adopted to provide or form a decorative means.

A second aspect of the invention is characterized by a kit comprising: a box, a plurality of the above bandages disposed within said box, one or more coloring markers disposed within said box, which are adapted to provide coloration to the decorative means of said bandages.

The above and other objects, effects, features, and advantages of the present invention will become more apparent from the following description of the embodiments thereof taken in conjunction with the accompanying drawings.

### BRIEF DESCRIPTION OF THE DRAWINGS

FIG. 1A is a plan view showing the upper side of the bandage.

FIG. 1B is a plan view showing the lower side of the bandage that is opposite the side shown in FIG. 1A

FIG. 1C is a cross-section view of the bandage shown in FIGS. 1A and 1B.

FIG. 2 is a partial cut way perspective view of the kit having bandage and markers to illustrate the contents thereof

FIGS. 3A, B and C are plan views showing the upper side of the bandage in various alternative embodiments.

### DETAILED DESCRIPTION

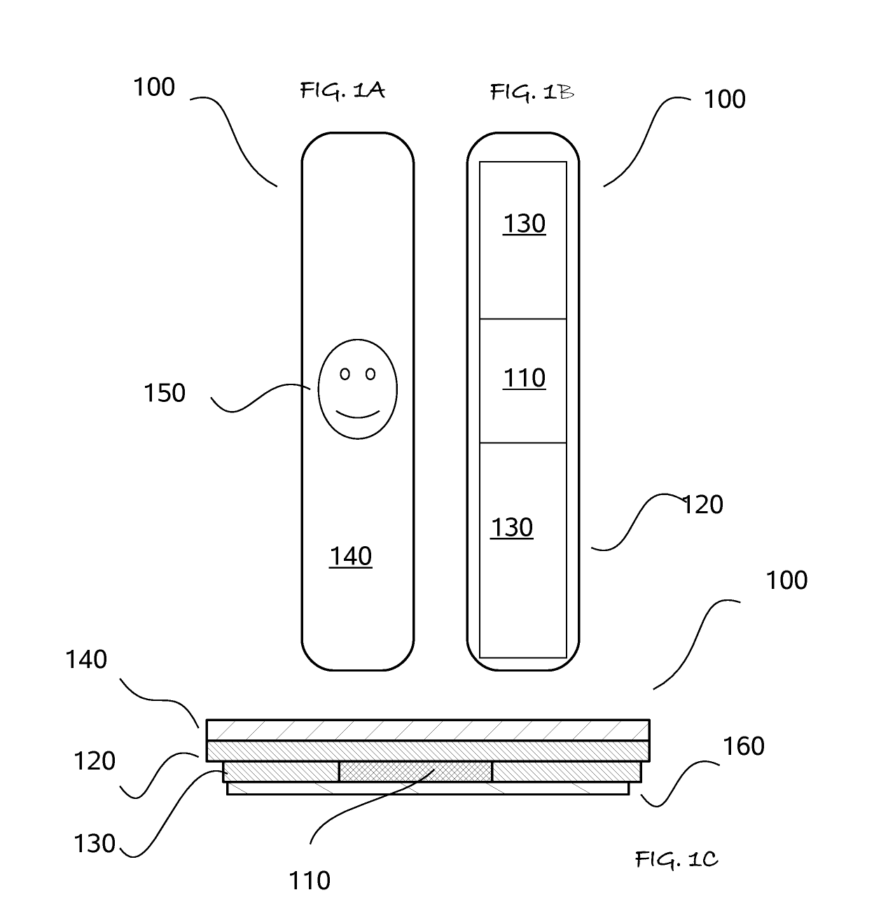

Referring to FIGS. 1 through 3, wherein like reference numerals refer to like components in the various views, there is illustrated therein a new and improved Decorative Adhesive Wound Care Systems, having components generally denominated 100 and 200 herein.

A primary component is a BANDAGE 100 having a generally planar absorbent layer 110 portion on one side. An outer base layer 120 expands beyond the edge on the absorbent layer. An adhesive layer 130 attached to and covering the portion of the base layer 120 that are on the same side as the absorbent layer that are not covered by the absorbent layer 110. An outer portion 140 on side of base layer 120 opposite that absorbent 110 and adhesive layers 130 that is adopted to provide or form a decorative means, in which decorative means include: a) an Outer surface of the base layer or a covering layer on the outer surface of the base layer is black for receiving a phosphorescent colorant; b) an Outer surface of the base layer or a covering layer on the outer surface of the base layer is adapted to receive and absorb a colorant and block the penetration of the colorant to the absorbent layer, c) Outer surface of the base layer or a covering layer on the outer surface of the base layer is has one or more visible a pre-printed outlines 150 (in addition to decorative means (b); d) an Outer surface of the base layer or a covering layer on the outer surface of the base layer is or contains a pre-printed thermo-chromic image (which is reversible or irreversible when heated to at least near body temperature, and e) an outer surface of the base layer or a covering layer on the outer surface of the base layer is or contains a pre-printed hydro-chromic image (which is reversible or irreversible when moistened). Bandages 100 optionally have a conventional pair of peelable release films 160 that cover the adhesive layer and the absorbing layer. It should be appreciated that all of the above layers are preferably generally thin, planar, and at least one of flexible and pliable. It should be noted that in the case of pre printed outlines 150, they preferably have a light grey color.

In such a bandage an incentive to cooperate in the process of therapeutic wound care by the reward of drawing or being entertained by a dynamic and transient image media the bandage 100 provides.

Another aspect of the invention is a kit 200 that comprises a box 210 with a plurality of bandages 100 contained therein, along with one or more coloring markers 220 adapted to provide coloration to the decorative means on the bandages.

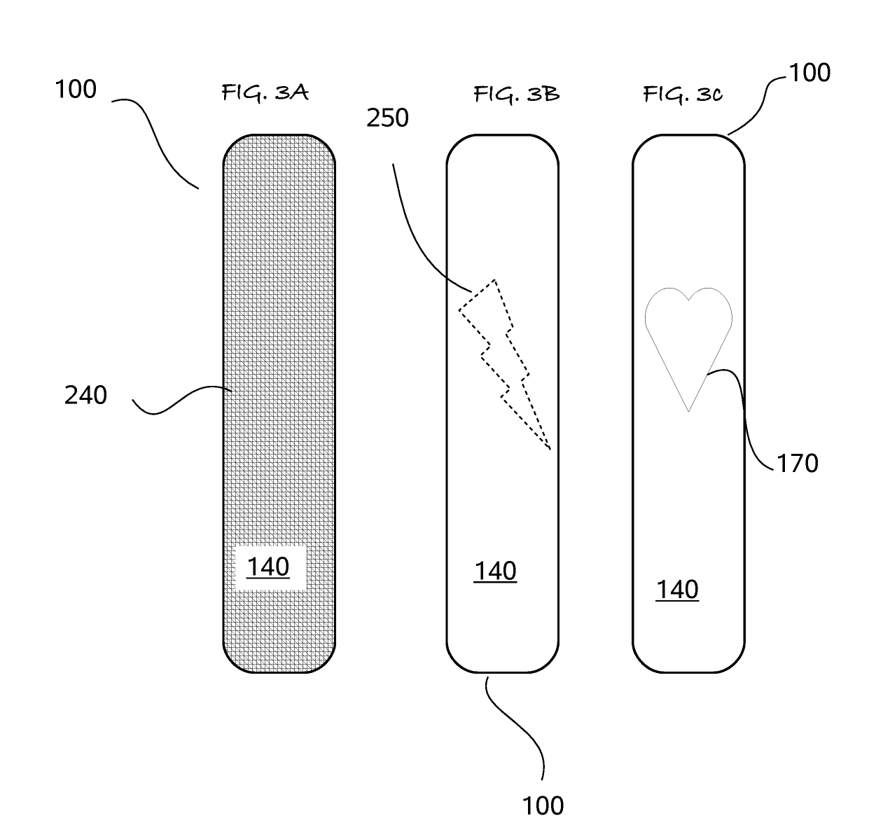

FIGS. 3A, B and C illustrate in further details the various decorative means, in particular in FIG. 3A, bandage 100 has an outer surface 140 of the base layer or a covering layer on the outer surface 240 of the base layer that is black for receiving a phosphorescent or fluorescent colorant, so that image will stand out in the dark after being exposed to light and will be visible in daylight as well. In FIG. 3B, the outer surface of the base layer or a covering layer on the outer surface of the base layer is or has one or more visible a pre-printed outlines 250, such as the broken line in the shape of a lightning bolt. Such preprinted lines are optionally, bold, faint, broken or solid lines. In FIG. 3C, the outer surface of the base layer or a covering layer on the outer surface of the base layer is or contains a pre-printed thermo-chromic image 170 or hydro-chromic image, which is visible in FIG. 3C. Such pre-printed, thermo or hydro-chromic images may represent any of pseudo-tattoos type graphics, cartoon characters, superhero characters and the like. Any such images may be combined in alternative combination of any 2, as well as all three types.

Another aspect of the invention is the method of using the bandages and kit described above, having the steps of a) removing a bandage from the box, b) Applying the coloring marker to the decorative means, and c) Applying the bandage to the skin/wound surface.

It should be appreciated that in this process, it is frequently preferable that step (c) is preferably before step (b).

Other aspects of the invention include having a colorant or ink that is applied by markers in the kit is preferably water based so easily cleanable from clothing, furniture, skin and the like surface, but is adapted to form a bond with the matter in the decorative layer so as to be non removable therefrom when exposed to water.

Colorant or ink that is applied by markers in the kit is preferably adapted to immediately absorb in the matter in the decorative layer so as to not smear after application thereto, and to not be readily removable therefrom.

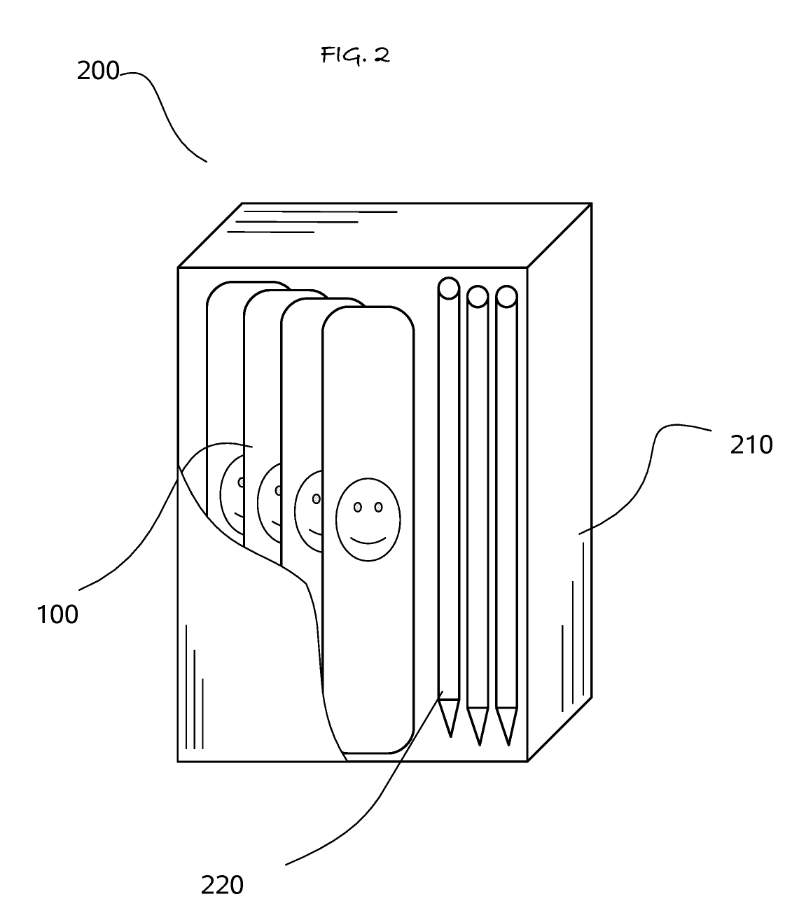

It should also be appreciated that in any of the embodiments described and illustrated above in which an outer surface of the base layer or a covering layer on the outer surface of the base layer is adapted to receive and absorb a colorant, it is also preferable that the combination of a preferred or recommended outer layer block the penetration of the colorant to the absorbent layer. This can be accomplished with the use of the kit 200 shown in FIG. 2, in which the colorant and bandage are selected to meet this objective, while the colorant also preferably bonds durably to the outer layer, but can also be washed with soap and water from skin and other surface it may incidentally contact. The same bandages and kits are alternatively used in clinical settings where important information such as dates and wound changes instructions are optionally marked on the outer layer.

It should be appreciated that the colorant or ink applied by the user to provide a thermo-chromic or hydro-chromic image. Alternatively, such an image can be pre-printed on the outside layer of the bandage cover.

Suitable thermochromic inks, paints and dyes for pre-marking the outside layer of the wound dressing are disclosed in the following U.S. Pat. Nos. which are incorporated herein by reference: 8,002,851; 6,669,765; 6,660,345; 6,139,779; 5,997,849; 5,705,093; 5,690,857 and 5,500,040. Such inks, paints and dyes may also be suitable for inclusion in marker, pen and crayon types devices so that the consumer can write or draw on the outside layer of the wound dressing.

Washable ink compositions for inks, paints and dyes may also be suitable for inclusion in marker, pen and crayon types devices so that the consumer can write or draw on the outside layer of the wound dressing are disclosed in the following U.S. Pat. Nos. which are incorporated herein by reference: 7,455,725; 6,458,193; 6,160,034; 5,874,144 and 5,486,228.

Suitable florescent or phosphorescent compounds and compositions for inclusion in inks, paints and dyes for pre-marking the outside layer of the wound dressing are disclosed in the following U.S. Pat. Nos. which are incorporated herein by reference: 8,026,306; 7,897,653; 7,837,777; 7,785,717; 7,464,965; 7,749,404; 7,674,326, 7,575,253; 7,470,731; 7,429,291; 7,364,613; 7,252,708; 7,018,564; 6,881,000; 6,749,773 and 6,400,386. Such florescent or phosphorescent inks, paints and dyes may also be suitable for inclusion in marker, pen and crayon type devices so that the consumer can write or draw on the outside layer of the wound dressing.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

I claim:

1) A bandage comprising:
a) a generally thin planar and flexible absorbent layer having an upper surface and an opposing lower surface,
b) a generally thin planar and flexible base layer having an outer surface and an opposing inner surface, wherein said base layer laterally expands beyond at least two edges of the absorbent layer, and further wherein the upper surface of the said absorbent layer is attached the inner surface of the base layer,
c) a generally thin planar and flexible adhesive layer attached to and covering the inner portions of the base layer that is not attached to the absorbent layer,
d) wherein the outer surface of said base layer opposite is adopted to provide or form a decorative means.

2) A bandage according to claim 1 wherein said decorative means is the outer surface of the base layer or a covering layer on the outer surface of the base layer that is black for receiving a phosphorescent colorant.

3) A bandage according to claim 1 wherein said outer surface of the base layer or a covering layer on the outer surface of the base layer is adapted to receive and absorb a colorant and block the penetration of the colorant to the absorbent layer.

4) A bandage according to claim 3 wherein said outer surface of the base layer or a covering layer on the outer surface of the base layer is adapted to receive and absorb a water washable colorant and block the penetration of the colorant to the absorbent layer.

5) A bandage according to claim 1 wherein the outer surface of the base layer or a covering layer on the outer surface of the base layer is has one or more visible pre-printed outlines.

6) A bandage according to claim 1 wherein the outer surface of the base layer or a covering layer on the outer surface of the base layer is or contains at least one pre-printed thermo-chromic image.

7) A bandage according to claim 1 wherein the at least one pre-printed thermo-chromic image changes appearance either reversibly or irreversibly when heated to at least near body temperature.

8) A bandage according to claim 1 wherein the outer surface of the base layer or a covering layer on the outer surface of the base layer is or contains a pre-printed hydro-chromic image which changes appearance either reversibly or irreversibly when moistened.

9) A kit comprising:
a) a box,
b) a plurality of bandages according to claim 1 disposed within said box,
c) one or more coloring markers disposed within said box, which are adapted to provide coloration to the decorative means of said bandages.

10) A kit according to claim 9 wherein the one or more coloring markers are selected from the group consisting of water washable markers, thermo-chromic marker, florescent markers, phosphorescent markers and thermochromic markers

11) A kit according to claim 9 wherein at least a portion of the bandages have an outer surface of the base layer or a covering layer on the outer surface of the base layer that is black and the one or more coloring markers are selected from the group consisting of florescent and phosphorescent markers.

12) A kit according to claim 10 wherein at least a portion of the bandages have an outer surface of the base layer or a covering layer on the outer surface of the base layer has one or more visible pre-printed outlines.

13) A kit according to claim 9 wherein at least a portion of the bandages have an outer surface of the base layer or a covering layer on the outer surface of the base layer is adapted to receive and absorb a colorant and block the penetration of the colorant to the absorbent layer and wherein the one or more coloring markers deploy a colorant that is particularly adapted to be permanently received by the outer surface of the base layer or a covering layer on the outer surface of the base layer.

14) A kit according to claim 13 wherein the one or more coloring markers deploy a colorant that is water washable when not received by the outer surface of the base layer or a covering layer on the outer surface of the base layer and forms a water fast image when received by the outer surface of the base layer or a covering layer on the outer surface of the base layer.

15) A process for marking the bandage of claim 1, the process comprising the steps of:
a) providing at least one bandage of claim 1,
b) providing a coloring marker,
c) applying the bandage to the skin/wound surface, and
d) applying the coloring marker to the decorative means.
